(19) United States
(12) Patent Application Publication
(10) Pub. No.: US 2009/0030506 A1
(43) Pub. Date: Jan. 29, 2009

(54) ENDOPROSTHESIS AND METHOD FOR MANUFACTURING SAME

(75) Inventors: Bjoern Klocke, Zurich (CH); Nina Adden, Nuernberg (DE); Ullrich Bayer, Karlsbad (DE)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/178,343

(22) Filed: Jul. 23, 2008

(30) Foreign Application Priority Data
Jul. 24, 2007 (DE) 10 2007 034 363.0

(51) Int. Cl. A61F 2/06

(52) U.S. Cl. 623/1.46; 623/1.15

(57) ABSTRACT

A stent with a basic mesh comprising an at least largely biodegradable material and a coating (30) arranged on the biodegradable material. The basic mesh is covered completely by a coating, except for at least one degradation area (23, 25, 32), whereby the at least one degradation area (23, 25, 32) is designed as a recess in the coating (30).

### PRIORITY CLAIM

This patent application claims priority to German Patent Application No. 10 2007 034 363.0, filed Jul. 24, 2007, the disclosure of which is incorporated herein by reference in its entirety.

### FIELD

The present disclosure relates to an endoprosthesis or implant, in particular, an intraluminal endoprosthesis, e.g., a stent, in a basic mesh comprising an essentially biodegradable material and a coating provided on the biodegradable material.

### BACKGROUND

Stents are endovascular prostheses that may be used for treatment of stenoses (vascular occlusions). Stents have a tubular or hollow cylindrical basic mesh which is open at both longitudinal ends. The tubular basic mesh of such an endoprosthesis is inserted into the blood vessel to be treated and serves to support the blood vessel.

Such stents have become well established for treatment of vascular diseases, in particular. Through the use of stents, constricted areas in blood vessels can be widened resulting in an increase in lumen diameter. Through the use of stents, an optimal vascular cross section can be achieved, and this is the primary requirement for therapeutic success; but the permanent presence of such a foreign body initiates a cascade of microbiological processes that may result in gradual adhesion of the stent and, in the worst case, a vascular occlusion. A starting point to solve this problem comprises manufacturing the stent from a biodegradable material.

For purposes of the present disclosure, the term “biodegradation” refers to hydrolytic, enzymatic and other metabolic degradation processes in the living body caused mainly by body fluids coming in contact with the endoprosthesis and leading to gradual dissolution of at least large portions of the endoprosthesis. For purposes of the present disclosure, the term “biocorrosion” is synonymous for the term “biodegradation.” For purposes of the present disclosure, the term “bioabsorption” includes subsequent absorption of the degradation products by the living body.

Materials suitable for the basic mesh of biodegradable endoprostheses may be of a polymeric or metallic nature, for example. The basic mesh may also comprise several materials. The common feature of these materials is their biodegradability. Examples of suitable polymeric compounds include polymers from the group of cellulose, collagen, albumin, casein, polysaccharides (PSAC), polylactide (PLA), poly-L-lactide (PLLA), polyglycol (PGA), poly-D,L-lactide-co-glycolide (PDLLA-PGA), polyhydroxybutyric acid (PHB), polyhydroxyvaleric acid (PHV), polyalkyl carbonates, polyorthoesters, polyethylene terephtalate (PET), polymalonic acid (PML), polyanhydrides, polyphosphazenes, polyamino acids and their copolymers as well as hyaluronic acid. The polymers may be used in pure form, in derivatized form, in the form of blends or as copolymers, depending on the desired properties. Metallic biodegradable materials are based on alloys of magnesium, iron, zinc and/or tungsten. The present disclosure preferably relates to stents or other endoprostheses in which the biodegradable material contains magnesium or a magnesium alloy, especially preferably the alloy WE43, and/or a biodegradable polymer, especially preferably PLLA.

Stents having coatings with various functions are already known in the art. In implementation of biodegradable implants, there is the problem of controlling the degradability according to the treatment desired. No stent has yet been found which loses its integrity within the target corridor of four weeks to six months, which is considered important for many therapeutic applications. For purposes of the present disclosure, the term “integrity,” i.e., mechanical integrity, refers to the property whereby the stent and/or the endoprosthesis undergoes hardly any mechanical losses in comparison with the undegraded stent. This means that the stent is still stable enough mechanically that the collapse pressure drops only slightly, i.e., to at most 80% of the nominal value. The stent can thus fulfill its main function, namely keeping the blood vessel open, while the integrity of the stent is preserved. As an alternative, integrity may be defined such that the stent is so stable mechanically that it is hardly subject to any mechanical changes in its load state in the blood vessel, e.g., does not collapse to any mentionable extent, i.e., under a load of at least 80% of the dilatation diameter, or it has supporting struts that have hardly been broken through at all.

Degradable magnesium stents have proven to be especially promising for the aforementioned target corridor of degradation, although the degradable magnesium stents lose their mechanical integrity and/or supporting effect too soon, and on the other hand, degradable magnesium stents have a highly fluctuating loss of integrity in vitro and in vivo. This means that, in the case of magnesium stents, the collapse pressure drops too rapidly over time and/or the drop in collapse pressure is subject to too great a variability and is, therefore, indeterminate.

There are essentially three known approaches to solving this problem. First, a thicker optimized stent design may be selected. Secondly, an optimized, slowly degrading magnesium alloy may be used for the stent. Thirdly, surface layers may be provided which delay or accelerate the degradation attack on the basic magnesium mesh and/or influence the point in time of the onset of degradation. The possibility of varying the degradation behavior according to the first or second possible approaches is greatly restricted and is perhaps not sufficient for an approach that is not economically and clinically satisfactory. With respect to the first possible case, wall thicknesses of more than 200 μm are not justifiable from the standpoint of guaranteeing easy insertability of the stent and the limited vascular dimensions. For the second case, only a very limited spectrum of biocompatible and moderately rapidly degradable alloys is known. With regard to the third possible case, only fluorine passivation is known.

The aforementioned passivation layers have two fundamental disadvantages resulting from the fact that such stents usually assume two states, namely a compressed state with a small diameter and an expanded state with a larger diameter. In the compressed state, the stent can be inserted into the blood vessel to be supported by using a catheter, and the stent can be positioned at the site to be treated. Then, at the site of treatment, the stent is dilated by means of a balloon catheter, for example, and/or (when using a memory alloy as the stent material) converted to the expanded state, e.g., by heating it to a temperature above the transition temperature. On the basis of this change in diameter, the basic mesh of the stent is subjected to a mechanical stress. Additional mechanical stresses on the stent may occur during production or in movement of the stent in or with the blood vessel into which the stent has been inserted. With the known passivation, this yields the disadvantage that microcracks occur during deformation of the implant leading to infiltration of the coating material thereby decreasing the passivation effect of the coating. This, in turn, causes nonspecific local degradation. Furthermore, the onset and speed of degradation depend on the size and distribution of the microcracks which are difficult to monitor as defects. This leads to a great scattering in the degradation times.

International Patent Publication No. WO2005/065576 discloses control of the degradation of degradable implants by means of a coating of a biodegradable material. Position-dependent degradation of the implant is optimized by the fact that the base body has an in-vivo position-dependent first degradation characteristic and has a coating of at least one biodegradable material covering the base body completely or optionally only in some areas, and the coating has a second degradation characteristic in vivo. The cumulative degradation characteristic at a given site is thus obtained from the sum of degradation characteristics of the material and the coating prevailing at the respective site. The position-dependent cumulative degradation characteristic is preselected by varying the second degradation characteristic, so that the degradation takes place at the defined location in a predetermined interval of time and with a predeterminable degradation course.

In International Patent Publication No. WO 2005/065576, the degradation characteristic of the biodegradable coating described there is achieved by varying the morphological structure of the coating, by substantive modification of the material and/or by adapting the layer thickness of the coating. “Morphological structure” is understood here to refer to the conformation and aggregation of the compounds forming the coating.

International Patent Publication No. WO 97/11724 also relates to a biodegradable implant and its degradation. This reference discloses that the degradation (disintegration) can be influenced by regulating the macroscopic structure of the biodegradable material, i.e., through different wall thicknesses, for example. The wall thickness of the implant at one end, the more slowly degrading end, is designed to be thicker than at the other end, the more rapidly degrading end, for example. This reference also indicates that the degradability may also be influenced by prehydrolysis or a change in crystallinity of the degradable material of the implant. In addition, this reference discloses the fact that by means of a corresponding biodegradable coating with a low water permeability, a change in degradation behavior can be accomplished.

U.S. Patent Publication No. 2006/0224237 also describes a transplant or a stent having a protective layer that is used to preserve surface structures of the stent from destruction. The surface structures here may be formed from one or more materials which are at least partially dissolved, degraded or absorbed in different environmental conditions.

The possibilities of influencing degradation mentioned in these references do not include any satisfactory solutions with regard to endoprostheses which degrade in the aforementioned target corridor. International Patent Publication No. WO 2005/065576 discloses only very general principles and does not provide any concrete proposed solutions with regard to magnesium stents in particular. International Patent Publication No. WO 97/11724 also preferably relates to polymer stents. In addition, due to the water permeability of the biodegradable coating, there are also problems in degradation due to infiltration and formation of gas bubbles under the cover layer.

U.S. Patent Publication No. US 2007/0050009 relates to a stent having a supporting structure of biodegradable material. This supporting structure is at least partially provided with an absorption inhibitor layer which reduces the rate of absorption of the supporting structure. The absorption inhibitor layer itself is also absorbed by the surrounding body fluids. By means of this approach known in the prior art, only very limited control of degradation of the stent is possible; but this is inadequate for many applications.

### SUMMARY

The present disclosure describes several exemplary embodiments of the present invention.

One aspect of the present disclosure provides an endoprosthesis, in particular, an intraluminal endoprosthesis, comprising a basic mesh comprising an at least largely biodegradable material and a coating arranged on the biodegradable material, the coating being inert, and the basic mesh is covered completely by the coating except for at least one degradation area for targeted control of degradation in the degradation area, whereby the at least one degradation area comprises a recess in the inert coating.

Another aspect of the present disclosure provides an endoprothesis, in particular, an intraluminal endoprothesis, having a basic mesh comprising an at least mostly biodegradable material and a coating arranged on the biodegradable material, wherein the coating contains at least parylene and the basic mesh is completely covered by the coating except for at least one degradation area for targeted control of degradation in the degradation area, whereby the at least one degradation area comprises a recess in the coating containing parylene.

A further aspect of the present disclosure provides a method for manufacturing an endoprosthesis, comprising a) providing the basic mesh of the endoprothesis with degradation elements; b) applying a coating containing at least parylene to the surface of the endoprosthesis so that it is completely covered; and c) treating the coating with oxygen plasma.

One aspect of the present disclosure provides an endoprosthesis whose mechanical supporting effect persists for a long period of time and whose degradation takes place at a controlled point in time, in particular, within the aforementioned target corridor. Furthermore, the degradation should be adapted to the geometric specifics of the stent design and the associated clinical requirements.

This aspect is achieved by an endoprosthesis whose coating is designed to be inert and whose basic structure is completely covered by the inert coating, except for at least one degradation area for targeted control of the degradation in this area, whereby the at least one degradation area is designed as a recess in the inert coating. For purposes of the present disclosure, the term “recess” refers to areas in the coating having a spatial extent of approximately 300 nm to max. almost to the extent of the endoprosthesis (e.g., 10 mm) and which expose the basic mesh and thus enable the molecules that are involved in the degradation, at least water, to reach the basic mesh.

For purposes of the present disclosure, the term “inert coating” refers to a layer which does not interact either chemically or biologically with the respective environment of the body being treated (physiological environment with a physiological pH), i.e., is essentially not absorbed by this environment, and which almost completely suppresses diffusion of water or other molecules and is thus essentially dense with respect to these molecules. Another property of the coating material is that the coating material does not swell to any mentionable extent. Degradation of the biodegradable material underneath the coating material is consequently also prevented by the inert coating. Furthermore, inert material is not thrombogenic and does not have any negative or pathological influence on the surrounding tissue and/or the surrounding body fluid.

The endoprosthesis of the present disclosure thus has degradation areas in the form of geometrically controlled recesses which very specifically expose only a portion of the surface of the endoprosthesis basic mesh and/or make it more readily attackable in a very targeted manner. The degraded material degrades more rapidly while in direct contact with body tissue and body fluid, e.g., blood. Ideally the geometry of the recesses, i.e., their geometric shape, is selected so that local degradation proceeds in an orderly and predictable manner. The desired loss of integrity is thus adjusted to the desired period of time, in particular, four weeks to six months.

The recesses can be created by using stencils or mechanical contacts in the coating during production of the endoprosthesis. To coat a stent completely except for the luminal side, for example, the stent may be pushed onto a cylindrical body (internal mandrel) with a slight inherent tension so the inside of the stent does not come in contact with the coating material during the coating operation. Through suitable surface structuring of the inside mandrel, the cylindrical body can be pulled out of the stent again at the end of the coating operation.

Starting from the degradation area, in degradation of an inventive endoprosthesis, the degrading endoprosthesis material is flushed out and there remains a thin tube of the inert coating, optionally of degradation products of the degraded endoprosthesis material. In the case of an endoprosthesis made of a magnesium alloy, this may results in soft magnesium degradation products and metabolic products, for example, such as calcium phosphate from the endogenous buffer system. In selecting suitable biocompatible alloys, the occurrence of such products is acceptable clinically.

The inert coatings have one or more polymers of the group, for example, polyphosphazenes, silicones, polyolefins, polyisobutylene, vinyl halide, polymers and copolymers, such as polyvinyl chloride, polyvinyl ether, such as polyvinyl methyl ether, polyvinyl ketones, polyvinyl aromatics, such as polystyrene or poly(styrene-isoprene-styrene), polyvinyl esters, such as polyvinyl acetate, copolymers of vinyl monomers with one another and olefins, such as ethylene-methyl methacrylate copolymers, acrylonitrile-styrene copolymers, polysulfone, poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), polyurethanes, polyisobutylene-poly(methyl methacrylate) and polydimethylsiloxane.

The inert coating may also be designed so that after degradation of the basic mesh the inert coating undergoes degradation in the basic environment, i.e., an environment having a higher pH than the physiological environment and formed by the degradation products of the endoprosthesis material. The degradation products here must be biocompatible with the pH-sensitive coating and need only occur in a small amount. For example, all Mg alloys degrade as a base, forming Mg ions. For example, when magnesium stents are degraded in the surface layers, the pH reaches levels of 10 to 11. Polymers suitable for such a coating include specific polyesters (e.g., selected polylactides) and polypeptides, which undergo alkaline decomposition at the aforementioned pH levels.

In an exemplary embodiment, the coating is additionally designed to be flexible, meaning that the coating follows the movement of the basic mesh so that no cracks or the like develop in the coating material. This means that the material of the coating itself does not have a supportive function, i.e., the coating is designed to be elastic and, therefore, flexible. This is the case even more when the basic mesh, which is situated beneath the coating, is degraded due to the degradation in the degradation areas, whereby the degradation also continues beneath the inert coating, starting from a degradation area arranged at the side. After complete degradation, the flexible coating, which does not have a supporting function itself, moves in a flexible manner with the blood vessel being treated in the wall of which it is typically embedded by endothelialization and partially also by proliferation of neointima and the body fluid flowing therein.

In another exemplary embodiment, the inert coating has one or more compounds from the group consisting of polysulfone, silicone rubber, polyurethane, diamond-like carbon (DLC) and synthetic glycocalix. These materials are especially suitable as a flexible inert coating and can be applied inexpensively. In addition to the aforementioned group of special organic substances, amorphous silicon carbide, magnesium phosphate and magnesium oxide are especially preferred inert coating materials, but they are only slightly flexible.

The above-described feature is also achieved by an endoprosthesis where the coating arranged on the surface of the basic mesh contains parylenes (which is a tradename for polyxylene polymers and is available from a number of commercial sources), preferably at least mainly parylenes, especially preferably parylene C or parylene N, whereby the basic mesh is completely covered by the coating except for at least one degradation area for targeted control of degradation in this area, whereby the at least one degradation area is designed as a recess in the coating. In the case of a coating with parylene, in particular, most preferably a coating which comprises at least 30 wt % parylene, preferably parylene C and/or parylene N, the degradation behavior is influenced in an especially positive manner. Parylene has, in particular, the properties of flexibility and low swelling volume described above.

For purposes of the present disclosure, parylenes are completely linear, partially crystalline and uncrosslinked aromatic polymers. These polymers can be divided into four different groups, namely parylene C, parylene D, parylene N and parylene F, depending on their structure.

Parylene is preferably applied by a plasma coating method. In an exemplary embodiment, the thickness of the coating with parylene, preferably the coating of parylene C or parylene N, in the areas different from the degradation areas is between approximately 0.1 μm and approximately 10 μm, preferably between approximately 0.4 μm and approximately 7 μm, especially preferably between approximately 1 μm and approximately 5 μm. With a layer thickness of more than 10 μm, the coating time is too long so the coating method becomes too expensive. Furthermore, a coating with a great thickness leads to a significant reduction in the lumen through which blood flows in the blood vessel of the patient treated (due to the induced production of neointima, among other things). With a layer thickness of less than 0.1 μm, inhomogeneities develop in the coating, based on the thickness of the layer, and defects also develop. Therefore, degradation of the basic mesh of the endoprosthesis beneath the coating is no longer reliably preventable and/or there is too much unwanted variability in the degradation that takes place.

Whereas completely closed parylene layers must subsequently be provided with recesses according to the present disclosure, parylene may also be applied in such thin layers (depending on the material and the properties of the surface of the basic mesh approximately 0.1 μm to 1 μm thick) that the layer is not closed but instead is in an island growth phase, and the recesses are formed from the surface areas where there is little or no coating.

In the areas provided with the inert and preferably flexible coating or the parylene-containing coating, the surface of the endoprosthesis is protected by the inert and biocompatible cover layer so that it survives mechanical stresses such as crimping, dilating or crossing of the lesion, in contrast with known passivation techniques, without developing cracks or other defects. In this way, uncontrolled degradation of the endoprosthesis in locations where it is not desired is prevented. By avoiding the development of cracks or defects in a flexible coating, a great scattering of degradation times can be prevented. In an exemplary embodiment, the degradation areas are arranged in the form of recesses so that the development of cracks is essentially prevented.

The degradation areas can be produced in various ways.

For example, laser radiation may be used for local ablation of the coating by thermal radiation in the infrared wavelength range. Especially Yb:YAG, Nd:YAG or CO₂ lasers as well as femtosecond lasers or fiber lasers are suitable for processing here.

Furthermore, electron radiation may be used to alter the chemical or physical properties of the coating locally and thermally. The electron beam source is preferably used in a vacuum chamber and especially preferably with a scanning unit with a multispot device. The scan unit with the multispot device allows a quasi-simultaneous heating of the endoprosthesis at several locations. For thermal modification of the coating, electromagnetic alternating fields may also be used.

As already indicated above, a coating containing parylene can preferably be applied, in particular, by means of a plasma coating method. Gas processes and electrolytic plasma processes may both be used. In this case, the plasma must be adjusted in a targeted manner for processing individual geometry segments of the endoprosthesis using suitable technical equipment (shielding, gas flow, backplate electrode shape, and the like) If necessary, the luminal side can be shielded in performing the plasma coating method, e.g., by pulling it onto silicone tube. In this method, the material partially migrates beneath the silicone tube during coating.

Ionic radiation may likewise be used for modification of the coating. The degradation areas may also be treated locally by bombarding with volatile solid bodies (e.g., dry ice), so the coating becomes brittle locally at the treated locations. Then the layer areas can be removed by other processes, e.g., by means of laser bombardment, electron bombardment or ionic bombardment. The degradation areas may also be created by bombardment with solid bodies (sand, ceramics, magnesium, salts, and the like) or liquids (water jet, oils, acid, fats) or solid body/liquid mixtures. Likewise, degradation areas can be created by mechanical machining of the layer (e.g., by means of needles, brush systems) in drums and/or by vibratory grinding methods (barrel finishing).

With the aforementioned production methods, corresponding lenses which are adapted to the corresponding endoprosthesis geometry may be used. In the case of laser bombardment, fiber optics may be used. In addition, highly dynamic handling techniques may be used and areas of coated endoprosthetic surface which are not to be machined can be shielded by means of masks.

With the especially preferred manufacturing method using a coating containing parylene with degradation areas, these areas are created by etching in an oxygen plasma following application of the coating. Coatings with parylene types C and N usually result in a macroscopically uniform covering on the surface of the endoprosthesis. Microscopically, however, there are layer thickness differences in the range of a few 0.1 μm with both layer variants, but distributed over the surface of the endoprosthesis.

If the surface of an endoprosthesis which is covered with a parylene coating preferably 1 to 5 μm thick is subjected to an oxygen plasma treatment, the coating is attacked by the oxygen ions. This results in a locally selective reduction in the parylene-containing coating. This loss of protective effect of the coating is inversely proportional to the layer thickness. If the process parameters (e.g., oxygen partial pressure, treatment time, chamber temperature) of the oxygen plasma are controlled in such a way that the coating is oxidized down to the base material at selected weak spots in the coating, then locally limited pinholes (recesses) that are free of coating and have a size of only a few 1 μm² are formed. The surface of these pinholes consists almost exclusively of the oxide of the endoprosthetic base material, preferably magnesium oxide. The surface of the pinholes is characterized by extensive absence of hydroxide in comparison with the surface of the endoprosthetic base material formed under atmospheric conditions. Therefore, the pinholes have a corrosion resistance that is lower than that of the coating but is greater than an untreated endoprosthetic surface. Therefore, the degradation attack which begins at these pinholes in subsequent use of the endoprosthesis under physical conditions in the body initially occurs with a delay and leads to corrosion of the base material after a partial conversion of the oxide to hydroxide. Since almost all pinholes of such an endoprosthesis have an identical surface composition, these locations are under uniform corrosive stress. Such a uniform and delayed local degradation constitutes the basis for a calculable integral endoprosthetic degradation. To etch the coating, the plasma etching, reactive ion etching and deep reactive ion etching methods may be used by analogy.

As another possibility, a resist can also be applied to the coating. This resist is structured so that only the coating is worn away at certain locations (intended breaking points) and subsequently is dissolved away again by wet chemical methods.

As another production variant, a special shaping/machining of degradable endoprosthesis is possible, creating the weak spots in the parylene layer to be applied later. In this exemplary embodiment, the recesses are not yet formed in the actual production of the endoprosthesis but instead are formed only in the course of implantation. These weak spots (intended breaking points) are formed, e.g., by holes and/or macropores in the webs of a stent that are created by laser cutting. This process step is used in the course of the usual laser cutting process. When the parylene coating is applied subsequently, it leads first to an effect of also sealing these intended breaking points. However, microcracks already develop, preferably in the vicinity of the zones with intended breaking points when the stent is dilated. These microcracks develop mainly in the regions around the intended breaking points, which have extremely high stress concentrations. The corrosion attack then takes place preferentially in these locations. The corrosion attack takes place uniformly over time and uniformly in the zones around the intended breaking point. The corrosion medium penetrates into the parylene layer, which is subject to microcracks, thereby leading to corrosion of the degradable material of the basic mesh beneath it, and ultimately leading to a weakening of the cross section of the stent webs due to attack by corrosion of an extent that can be calculated accurately per week. Alternatively, intended breaking points are formed in the parylene layer at those locations in the stent which undergo great deformation due to crimping and dilatation at the surface even due to the construction itself.

In another exemplary embodiment of the present disclosure, an adhesive layer is arranged between the inert coating and the material of the basic mesh which is not arranged in the area of the recesses of the degradation area. Such an adhesive layer improves the adhesion between the inert coating and the material of the basic mesh. Such an adhesive layer may contain, for example, one or more compounds from the group comprising magnesium oxide, magnesium phosphate, and inorganic magnesium compounds.

In a design of the endoprosthesis comprised of supporting elements, which are preferably designed with a zigzag, meandering or spiral pattern and which assume the function of supporting the blood vessel or other hollow organs and of connecting webs, connecting webs which connect the supporting elements but which do not themselves assume any supportive function, a plurality of degradation areas is arranged only in the area of the connecting webs in another exemplary embodiment. For example, one recess each is arranged only in the middle of a connecting web. Such an exemplary embodiment is especially simple in design and may also be implemented at a low production cost. Such an endoprosthesis has the advantage that its collapsing pressure drops very rapidly after a desired period of time, such as four weeks to six months. Such an endoprosthesis is especially desired for clinical use.

Preferably at least one degradation element, which protrudes away from the basic mesh essentially like an extension and has at least one degradation area, is provided on the basic mesh. Such degradation elements are especially simple to manufacture when they are designed to be essentially finger-shaped. However, a degradation element may also have a different shape. Such a degradation element preferably does not have any other functions except for the function of controlling degradation and, in particular, the degradation element has no mechanical function with respect to the endoprosthesis. According to the present disclosure, the degradation element is preferably made of the same material as the basic mesh and also has the complete coating of flexible inert material with the same layer thickness as that on the basic mesh except in the degradation areas. In this way, the degradation element, as part of the stent, can be produced easily together with the basic mesh, e.g., by laser cutting.

The degradation element also extends essentially in the area of the jacket volume formed by the basic mesh. This jacket volume is the outer jacket area of the cylinder formed by the endoprosthesis. If the degradation element does not protrude inward radially out of this jacket area, additional unwanted turbulence in the body fluid flowing in the blood vessel provided with the endoprosthesis is prevented. It is also not desirable for the degradation element to protrude outward because otherwise the degradation element would penetrate through the blood vessel or hollow organ in which the endoprosthesis is arranged.

In another exemplary embodiment, each degradation element has exactly one degradation area which is arranged on the end of the degradation element protruding away from the basic mesh. This means that, for example, the end protruding away from the basic mesh does not have an inert coating and is therefore exposed. In this way, the degradation begins at this end of the degradation element due to the thickness and length as well as the arrangement of the degradation element on the basic mesh. It is possible to control when and where the basic mesh is then degraded.

To achieve the desired degradation times in the target corridor of four weeks to six months, the degradation elements in one exemplary embodiment have a diameter of approximately 50 μm to approximately 200 μm. In the case of a stent, it is preferable in terms of manufacturing technology if the degradation elements have the same geometry and/or thickness as the stent struts. It is also preferable if the degradation elements have a length of up to approximately 0.3 mm, preferably approximately 0.1 mm. The length of the degradation elements and their thickness are measured without taking into account the inert coating. In selecting the dimensions of the degradation element, it is important that the degradation elements do not contact the stent struts even when the stent is crimped onto the catheter. This prevents the coating from being damaged (scratched) in crimping.

In another exemplary embodiment, the degradation area is designed as a ring-shaped recess, which extends around a supporting element, a connecting web or a degradation element. In additional exemplary embodiments, the recess may also have other forms which extend completely or partially around the elements mentioned hereinabove. In another exemplary embodiment, the degradation area is designed as a circular recess, a polygonal recess or a recess having any other conceivable shape. Such recesses especially preferably have a diameter of approximately 1 to 10 μm. Such exemplary embodiments of degradation areas can be produced especially easily and inexpensively. In addition, recesses with straight edges are preferred.

Especially preferably at least one degradation area is provided on a plurality of connecting webs arranged in a predetermined area of the endoprosthesis and/or on the degradation elements arranged on one of these connecting webs. The connecting webs here together with the supporting elements form the basic mesh. The basic mesh comprises supporting elements which are arranged axially in succession and run essentially in the circumferential direction and connecting webs which connect the individual supporting elements. In this way, the connecting webs are degraded first so that the flexibility with regard to the blood vessel is optimized at a point in time soon after insertion of the endoprosthesis. The integrity of the ring-shaped supporting elements which provide the support is maintained for a longer period of time, i.e., as long as is clinically necessary.

### BRIEF DESCRIPTION OF THE DRAWINGS

Various aspects of the present disclosure are described hereinbelow with reference to the accompanying figures.

The present disclosure is explained in greater detail below on the basis of exemplary embodiments depicted in the figures. All the features described here and/or illustrated in the figures form the subject matter of the present disclosure, regardless of how they are combined in the claims or their reference back to previous claims.

FIG. 1 shows a cross-section view of a first exemplary embodiment of an endoprosthesis;

FIG. 2 shows a cross-section view of a second exemplary embodiment of an endoprosthesis;

FIG. 3 shows a side cross-section of a third exemplary embodiment of an endoprosthesis;

FIG. 4a shows the structural formula of parylene C

FIG. 4b shows the structural formula of parylene N;

FIG. 5 shows a side view of a fourth exemplary embodiment of an endoprosthesis.

### DETAILED DESCRIPTION

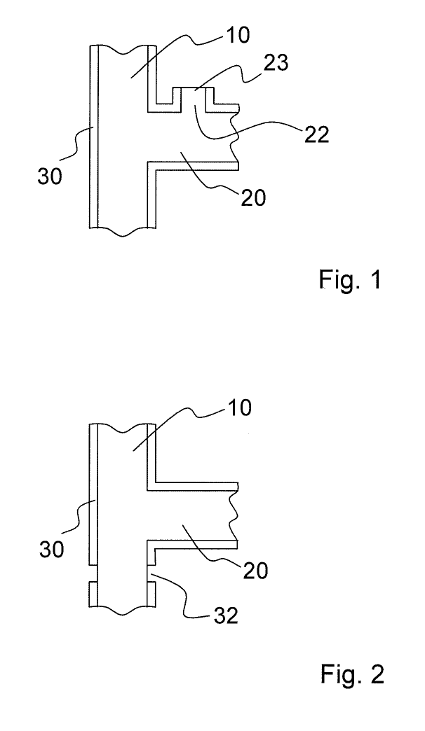

FIG. 1 shows a section through a basic mesh of an endoprosthesis according to one exemplary embodiment which is designed as a stent. The basic mesh has webs that are folded in a zigzag or meandering pattern, running essentially in the circumferential direction, or helical webs as the supporting elements 10 as well as webs running essentially in the longitudinal direction of the stent as connecting webs 20. The stent is designed as a whole as a tubular or cylindrical endoprosthesis running in the direction of the connecting webs 20 designed to be open at its ends. FIG. 1 shows only a section of the basic mesh in which the end of a connecting web 20 abuts against a supporting element 10.

The basic mesh of the stent comprises at least primarily one or more largely biodegradable materials and on its entire surface, not only in the degradation areas described in detail hereinbelow, has an inert flexible coating 30 that completely covers the basic mesh and has an essentially constant layer thickness. Conventional biodegradable materials, in particular, are mentioned hereinabove. Parylene C or parylene N or polysulfone, for example, may be considered as materials for the coating 30.

The exemplary embodiment depicted in FIG. 1 has a degradation element 22 in the form of a finger-shaped extension on a plurality of connecting webs 20. The finger-shaped extension 22 has an essentially cylindrical shape, which in another exemplary embodiment (not shown in FIG. 1) may taper in the direction pointing away from the basic mesh, i.e., with its diameter being reduced.

On an end 23 protruding away from the connecting web 20, the degradation element 22 does not have an inert coating 30. The material of the stent is exposed here so that the body fluid can act directly on the exposed degradable material of the finger-shaped extension and cause degradation of this material. The end 23 of the finger-shaped extension 22 is thus a degradation area in which degradation of the stent begins. In the other areas, the inert coating 30 prevents degradation.

The exemplary embodiment of a stent shown in FIG. 2 has no finger-shaped extension 22 on the part of the connecting web 20. Instead, a ring-shaped recess 32 running around the supporting element 10 is provided in the area of the supporting element 10 so that the degradable material of the supporting element 10 is also exposed for attack by degradation. Through such a recess, it is possible to control accurately where the degradation of the stent begins.

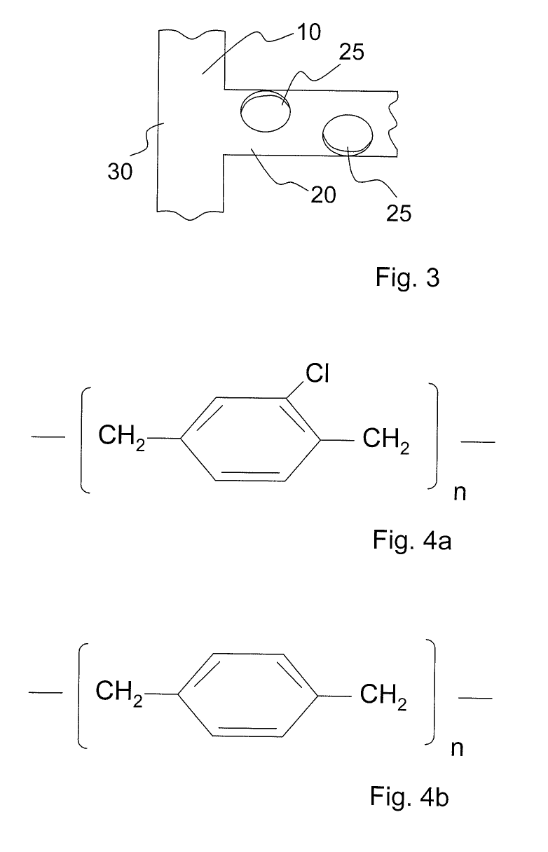

In a third exemplary embodiment depicted in FIG. 3, the degradation areas are implemented by circular areas 25 arranged preferably on the connecting webs 20 with the inert coating 30 recessed completely in these areas and the degradable material being exposed there. The degradation of the stent will also begin in these locations after the stent is inserted into the body.

The exemplary embodiments shown herein for the arrangement of the degradation areas may be varied at will according to the desired degradation behavior. Consequently, the finger-shaped extensions 22 may also be arranged on the supporting elements 10 or in other locations on the connecting webs 20. Furthermore, the finger-shaped extensions 22 may also be arranged in multiple locations on the supporting elements or only on certain supporting elements 10 and/or connecting webs 20. This also applies to the ring-shaped recess 32 or the circular area 25. The various types of degradation areas may also be combined at will on one endoprosthesis (even with the degradation elements).

The endoprostheses may be produced from the biodegradable material by first producing the endoprosthesis by known manufacturing methods. If necessary, the finger-shaped extensions 22 or other degradation elements are provided here at the desired locations on the basic mesh. Next, the coating 30 is applied by means of known coating methods (e.g., for parylene by means of a plasma coating method) whereby a cover is provided before the coating at the locations where a degradation area is to be provided so that the coating is not applied in these areas during the coating process. Stencils may be used instead for this purpose. Next the covering is removed. Alternatively, the coating may also be applied first to the entire surface of the endoprosthesis (including any degradation elements applied, if necessary) and then at least partially removed in the degradation areas. In the case of the degradation area arranged on the end 23 of a finger-shaped extension 22, this may happen, for example, by a part of the end being cut off.

Parylene C and parylene N, the chemical structures of which are shown in FIG. 4a and FIG. 4b, respectively, are preferred materials of the inert coating 30,

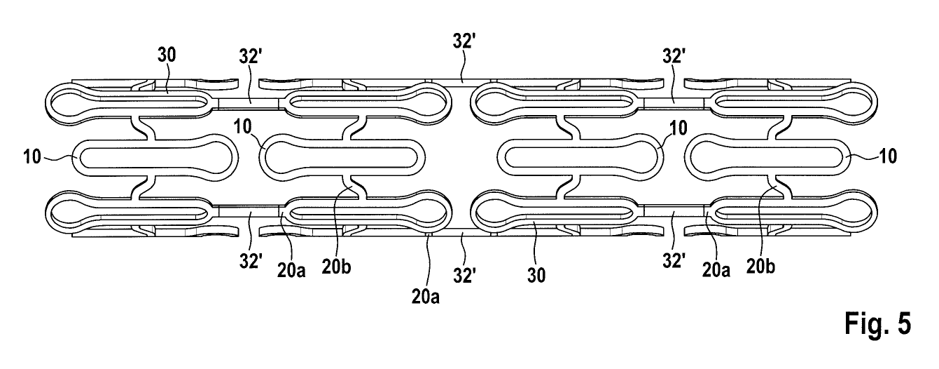

FIG. 5 shows again a longer section of an endoprosthesis according to the present disclosure in the form of a stent having ring-shaped and peripheral recesses 32′ in the coating 30 on the connecting webs 20a of the supporting elements 10 running in the longitudinal direction, said recesses extending over almost the entire length of these connecting webs. The degradable material is exposed in the area of these recesses, which are shown in FIG. 5. The connecting webs 20b of the supporting elements 10, which do not run in the longitudinal direction but instead are curved essentially in a radial direction, do not have any recesses.

All patents, patent applications and publications referred to herein are incorporated by reference in their entirety.

What is claimed is:

1. An endoprosthesis, in particular, an intraluminal endoprosthesis, comprising:
a basic mesh comprising an at least largely biodegradable material and a coating arranged on the biodegradable material, the coating being inert, and the basic mesh is covered completely by the coating except for at least one degradation area for targeted control of degradation in the degradation area, whereby the at least one degradation area comprises a recess in the inert coating.

2. The endoprosthesis of claim 1, wherein the inert coating is flexible.

3. The endoprosthesis of claim 1, wherein the inert coating comprises one or more compounds from the group consisting of polysulfone, silicone rubber, polyurethane, synthetic glycocalix, amorphous silicon carbide, diamond-like carbon (DLC), magnesium phosphate, magnesium oxide and mixtures of the foregoing.

4. The endoprothesis of claim 1, further comprising an adhesive layer arranged between either the inert coating or the coating that contains parylene and the material of the basic mesh, and wherein the adhesive layer contains one or more compounds from the group consisting of magnesium oxide, magnesium phosphate and inorganic magnesium compounds.

5. The endoprosthesis of claim 1, wherein the at least one degradation area comprises a plurality of degradation areas which are arranged only proximate to the connecting webs.

6. The endoprothesis of claim 1, the basic mesh further comprising at least one degradation element which protrudes away from the basic mesh, each degradation element having at least one degradation area, the degradation element having a finger shape and extending essentially in the area of the jacket volume formed by the basic mesh.

7. The endoprothesis of claim 6, wherein each degradation element has exactly one degradation area which is arranged on the end of the degradation element that protrudes away from the basic mesh.

8. The endoprothesis of claim 6, wherein each degradation element has a diameter of approximately 50 to approximately 200 μm and a length of up to approximately 0.3 mm.

9. The endoprothesis of claim 1, wherein the at least one degradation area comprises a ring-shaped recess which extends around either a supporting element or a connecting web and the at least one degradation area comprises either a circular or a polygonal recess in the coating.

10. The endoprothesis of claim 1, the basic mesh further comprising a plurality of supporting elements which run essentially in the circumferential direction and are arranged one after the other in the axial direction, with connecting webs that connect the individual supporting elements, whereby at least one degradation area is provided on a plurality of connecting webs arranged in a predetermined area of the endoprosthesis and on the degradation elements arranged on one of the connecting webs.

11. The endoprothesis of claim 1, wherein the biodegradable material comprises a material selected from the group consisting of Mg, Mg alloy, WE43, a biodegradable polymer, and PLLA.

12. The endoprothesis of claim 1, wherein the coating further comprises one or more polymers of the group consisting of polyesterspolylactides and polypeptides.

13. An endoprothesis, in particular, an intraluminal endoprothesis having a basic mesh, comprising: an at least mostly biodegradable material and a coating arranged on the biodegradable material, wherein the coating contains at least parylene and the basic mesh is completely covered by the coating except for at least one degradation area for targeted control of degradation in the degradation area, whereby the at least one degradation area comprises a recess in the coating containing parylene.

14. The endoprothesis of claim 13, wherein the layer thickness of the coating containing parylene in the areas which are different from the degradation areas is between approximately 0.1 μm and approximately 10 μm.

15. The endoprothesis of claim 13, further comprising an adhesive layer arranged between the inert coating or the coating that contains parylene and the material of the basic mesh, the adhesive layer containing one or more compounds from the group consisting of magnesium oxide, magnesium phosphate and inorganic magnesium compounds.

16. The endoprosthesis of claim 13, wherein a plurality of degradation areas is arranged only in the area of the connecting webs.

17. The endoprothesis of claim 13, the basic mesh further comprising at least one degradation element which protrudes away from the basic mesh each degradation area having at least one degradation area, the degradation element having a finger shape and extending essentially in the area of the jacket volume formed by the basic mesh.

18. The endoprothesis of claim 13, wherein each degradation element has exactly one degradation area which is arranged on the end of the degradation element that protrudes away from the basic mesh.

19. The endoprothesis of claim 13, wherein the degradation element has a diameter of approximately 50 to approximately 200 μm and the degradation element has a length of up to approximately 0.3 mm.

20. The endoprothesis of claim 13, wherein the at least one degradation area has a ring-shaped recess which extends around either a supporting element or a connecting web and the at least one degradation area has either a circular or a polygonal recess in the coating.

21. The endoprothesis of claim 13, the basic mesh further comprising supporting elements which run essentially in the circumferential direction and are arranged one after the other in the axial direction, with connecting webs that connect the individual supporting elements, whereby at least one degradation area is provided on a plurality of connecting webs arranged in a predetermined area of the endoprosthesis and on the degradation elements arranged on one of these connecting webs.

22. The endoprothesis of claim 13, wherein the biodegradable material contains a material selected from the group consisting of Mg, Mg alloy, WE43, a biodegradable polymer, and PLLA.

23. The endoprothesis of claim 13, wherein the coating contains at least one polymer from the group consisting of polyesters, polylactides and polypeptides.

24. A method for manufacturing an endoprosthesis, comprising:
a) providing the basic mesh of the endoprothesis with degradation elements;
b) applying a coating containing at least parylene to the surface of the endoprosthesis so that it is completely covered; and
c) treating the coating with oxygen plasma.

25. The method of claim 24, wherein the coating containing parylene is approximately 0.1 μm to approximately 10 μm thick is applied.

26. The method of claim 24, wherein the coating containing parylene is approximately 0.4 μm to approximately 7 μm thick.

27. The method of claim 24, wherein the coating containing parylene is approximately 1 μm to approximately 5 μm thick.

28. The endoprothesis of claim 6, wherein each degradation element has a diameter of approximately 50 to approximately 200 μm and a length of up to approximately 0.1 mm.

29. The endoprosthesis of claim 9, wherein the either circular or polygonal recess has a diameter of from approximately 1 to 10 μm.

30. The endoprothesis of claim 13, wherein the layer thickness of the coating containing parylene in the areas which are different from the degradation areas is between approximately 0.4 μm and approximately 7 μm.

31. The endoprothesis of claim 13, wherein the layer thickness of the coating containing parylene in the areas which are different from the degradation areas is between approximately 1 μm and approximately 5 μm.

32. The endoprothesis of claim 19, wherein each degradation element has a diameter of approximately 50 to approximately 200 μm and a length of up to approximately 0.1 mm.

33. The endoprosthesis of claim 20, wherein the either circular or polygonal recess has a diameter of from approximately 1 to 10 μm.
